(19) United States
(12) Patent Application Publication
(10) Pub. No.: US 2023/0095428 A1
(43) Pub. Date: Mar. 30, 2023

(54) [6R]-MTHF IN 5-FU BASED CHEMOTHERAPY OF BRAF- OR KRAS-MUTATED COLORECTAL CANCER

(71) Applicant: Isofol Medical AB, Göteborg (SE)

(72) Inventors: Roger Ingvar Tell, Saltsjö-Boo (SE); Lisa Katarina Skintemo, Lerum (SE); Per Emil Jonathan Holmén, Göteborg (SE); Per Lennart Lindberg, Göteborg (SE)

(21) Appl. No.: 17/488,139

(22) Filed: Sep. 28, 2021

(51) Int. Cl. A61K 31/282; A61P 35/04; A61K 31/4745; A61K 31/513; A61K 31/519; A61K 39/395

(52) U.S. Cl. CPC A61K 31/282; A61P 35/04; A61K 31/4745; A61K 31/513; A61K 31/519; A61K 39/3955

(57) ABSTRACT

The present invention relates to the treatment of colorectal cancer in human populations having a high frequency of BRAF- or KRAS-mutations, which involves administering multiple boluses of [6R]-5,10-methylenetetrahydrofolate ([6R]-MTHF) in connection with 5-fluorouracil (5-FU) based chemotherapy.

The instant application claims the benefit of priority under 35 U.S.C. § 119 to International Application No. PCT/EP2021/076512, filed Sep. 27, 2021, which is incorporated herein by reference in its entirety.

### FIELD OF THE INVENTION

The present invention relates to the treatment of solid tumors in humans with BRAF- or KRAS-mutated colorectal cancer or metastatic colorectal cancer, which comprises the administration of [6R]-5,10-methylene tetrahydrofolate (6R-MTHF) in 5-fluorouracil (5-FU) based chemotherapy, in combination with oxaliplatin or irinotecan.

### BACKGROUND OF THE INVENTION

Colorectal cancer (CRC) is one of the leading causes of mortality and morbidity in the world. With approximately 1,849,518 new cases estimated and 880,792 deaths per year (Caputo 2019), it also represents the third most common cancer worldwide and the second cause of cancer-related mortality, after lung cancer. In terms of geographical distribution, CRC incidence and prevalence have risen in industrialized countries (Bray 2018). Colorectal cancer affects approximately 135.439 estimated new patients in the United States per year. Of these cases, 39.910 per year (30%) are due to rectal cancer (Recio-Boiles 2020). However, in recent years the incidence and mortality rates of CRC have grown higher in Eastern Europe, Latin America, and Asia than other countries.

While the 5-year survival rate is 90% for early-stage CRC patients with localized disease, it is 70% for intermediate (regional invasive tumors) and only about 10% for advanced-stage patients with distant metastasis. Several factors including age, diet, hereditary polyposis syndrome and inflammatory bowel disease are associated with the development of CRC (Brenner 2015).

However, CRC is not a single type of tumor; its pathogenesis depends on the anatomical location of the tumor and differs between right side and left side of the colon. Further, CRC is a multigenetic disease, as several oncogenes are involved frequently in this process. Oncogenic mutations spread differently, based on specific anatomical regions.

Using bioinformatics analysis, Huang and coworkers (Huang 2018) identified six key driver genes, namely APC, KRAS, BRAF, PIK3CA, SMAD4 and p53. Through a systematic search, 120 articles published by Nov. 30, 2017 were included, which all showed roles for these gene mutations in CRC metastasis. A meta-analysis showed that KRAS mutations (combined OR 1.18, 95% CI 1.05-1.33) and p53 mutations (combined OR 1.49, 95% CI 1.23-1.80) were associated with CRC metastasis, including lymphatic and distant metastases. Moreover, CRC patients with a KRAS mutation (combined OR 1.29, 95% CI 1.13-1.47), p53 mutation (combined OR 1.35, 95% CI 1.06-1.72) or SMAD4 mutation (combined OR 2.04, 95% CI 1.41-2.95) were at a higher risk of distant metastasis. Subgroup analysis stratified by ethnic populations indicated that the BRAF mutation was related to CRC metastasis (combined OR 1.42, 95% CI 1.18-1.71) and distant metastasis (combined OR 1.51, 95% CI 1.20-1.91) in an Asian population (FIG. 1). No significant association was found between mutations of APC or PIK3CA and CRC metastasis. It was thus concluded that mutations of KRAS, p53, SMAD4 and BRAF play significant roles in CRC metastasis and may be both potential biomarkers of CRC metastasis as well as therapeutic targets.

BRAF and KRAS both belong to a class of genes known as oncogenes. When mutated, oncogenes have the potential to cause normal cells to become cancerous. B-Raf is a protein encoded by the BRAF gene and is involved in the RAS/MAPK pathway, which regulates cellular growth and division. BRAF mutations are found in 8-12% of cases of mCRC, with the predominance of BRAFV600E in approximately 90% of BRAF-mutant CRC. BRAFV600E is a point mutation at nucleotide 1799 that results in independent activation of its upstream activator protein, RAS, as well as increased stimulation of its downstream effector proteins, MEK and ERK, via phosphorylation. RAS and BRAF mutations are usually mutually exclusive (Tabernero 2020).

The KRAS gene encodes for a protein called K-Ras that is also part of the RAS/MAPK pathway. KRAS mutation rates are different among CRC patients belonging to different ethnicities. In Caucasians, the frequency of CRC with KRAS mutations is equal to 38%; in Asians, it is close to 40%; and, in Africans, it is only 21%. Most KRAS mutations in CRC occur within KRAS exon 2, with prevalence ranging from 10% to 47%, typically in codons 12 and 13. In addition, approximately 10% of patients with CRC are characterized by other types of KRAS mutations. Mutations in the KRAS gene are detected in approximately 25% of all human cancers; rates are highest in pancreatic carcinoma, reaching above 80%, while, in CRC, they are found in about 40% of cases (Cefali 2021).

In terms of treatment, the KRAS mutation has been found to have an adverse impact on the prognosis for stage IV CRC patients treated with the FOLFOX regimen (Zocche 2015) or on stage II or III CRC patients treated with adjuvant FOLFOX (Lee 2014). In a study related to first-line treatment of mCRC, there was no evidence for patients whose tumors carried mutations in KRAS of a benefit associated with the addition of cetuximab to FOLFIRI in relation to PFS, overall survival, or best overall response (ORR). Patients in both treatment groups whose tumors carried mutations in KRAS appeared to have worse overall survival than those whose tumors were wild-type. The addition of cetuximab to FOLFIRI in patients with KRAS wild-type disease resulted on the other hand in significant improvements in overall survival (Van Cutsem 2011). KRAS mutations have further been found to confer resistance to epidermal growth factor receptor (EGFR) inhibitors, a class of tyrosine kinase inhibitors or monoclonal antibodies designed to slow or halt uncontrolled cell growth (Bai 2015).

CRC patients with BRAF-mutations do not benefit, either, from EGFR inhibitors. The current standard therapy in first-line treatment of BRAF-mutated CRC is folate/5-fluorouracil (5-FU) assisted oxaliplatin-based chemotherapy plus bevacizumab, increasingly including the more intensive “triplet” therapy FOLFOXIRI (i.e. leucovorin/5-FU/oxaliplatin/irinotecan) plus bevacizumab, which however is only a valid option in patients with a good ECOG (Eastern Cooperative Oncology Group) performance status (Caputo 2019). Triplet chemotherapy has thus been found to be a very good option for otherwise fit CRC patients with aggressive tumors, and has also been suggested for KRAS-mutated CRC cases (Glynne-Jones 2020).

However, FOLFOXIRI plus bevacizumab triplet therapy is in general associated with a highly increased level of adverse side effects. In a meta-analysis of patient data from 5 clinical studies it was thus found that—compared with patients receiving doublet therapy (FOLFOX or FOLFIRI)—patients in the triplet group (FOLFOXIRI plus bevacizumab) had higher rates of grade 3 or 4 neutropenia (45.8% vs 21.5%, P<0.001), febrile neutropenia (6.3% vs 3.7%, P=0.019), nausea (5.5% vs 3.0%, P=0.016), mucositis (5.1% vs 2.9%, P=0.024), and diarrhea (17.8% vs 8.4%, P<0.001). Death due to toxicity occurred in 2.3% vs 1.4% of patients (P=0.277) (Cremolini 2020).

FOLFOXIRI plus bevacizumab triplet therapy therefore remains recommended only for patients whose general condition is very good, estimated to around 40-50% of all mCRC patients; discounting the elderly (Cremolini 2019).

Sadly, it is not the majority of mCRC patients who are sufficiently fit or young enough to be subjected to FOLFOXIRI plus bevacizumab triplet therapy. For the majority of patients with BRAF- and/or KRAS-mutated CRC, in particular metastatic CRC, there remains an unmet need for an improved folate-enhanced 5-FU treatment protocol of their disease.

### Definitions

As used herein, the term Leucovorin® or folinic acid shall both mean 5-formyl tetrahydrofolic acid, i.e. the 5-formyl derivative of tetrahydrofolic acid. Folinic acid contains 2 asymmetric centers. Commercially available leucovorin (LV) is composed of a 1:1 mixture of the dextrorotary and levorotary diastereomers (d-leucovorin (d-LV, (6R,2'S)-configuration) and 1-leucovorin (1-LV, (6S,2′S)-configuration), respectively), and may also be referred to as (d,1-LV).

As used herein, the term Levoleucovorin shall refer to the commercially available product which contains only the pharmacologically active levo-isomer 1-LV (or LLV). In vitro, 1-LV has been shown to be rapidly converted to the biologically available methyl-tetrahydrofolate form while the dextro form d-LV (DLV) is slowly excreted by the kidneys. Leucovorin and levoleucovorin have however been shown to be pharmacokinetically identical and may be used interchangeably with limited differences in efficacy or side effects (Kovoor et al, Clin Colorectal Cancer 8 200-6 (2009).

As used herein, the terms MTHF or methyleneTHF shall both refer to 5,10-Methylene-5,6,7,8-tetrahydrofolate.

As used herein, the terms racemic methyleneTHF, CoFactor® or [6R,S]-5,10-methyleneTHF shall all refer to the 1:1 diastereomeric mixture [6R,S]-5,10-Methylene-5,6,7,8-tetrahydrofolate.

As used herein, the term [6R]-5,10-MTHF shall refer to the single diastereomer, [6R]-5,10-methylenetetrahydrofolate.

As used herein, the terms IV or i.v. shall both mean intravenous.

As used herein, the term ORR shall refer to the Objective Response Rate, i.e. the proportion of patients with a reduction in tumor burden of a predefined amount.

This shall be calculated as follows: ORR=Sum of partial responses plus complete responses as per RECIST 1.1 (a set of published rules (link: https://recist.eortc.org/recist-1-1-2/) that define when tumors in cancer patients progress during treatments, the responses being defined as:

Complete Response (CR):

Disappearance of all target lesions. Any pathological lymph nodes (whether target or non-target) must have reduction in short axis to <10 mm.

Partial Response (PR):
At least a 30% decrease in the sum of diameters of target lesions, taking as reference the baseline sum diameters.

Progressive Disease (PD):
At least a 20% increase in the sum of diameters of target lesions, taking as reference the smallest sum on study (this includes the baseline sum if that is the smallest on study).
In addition to the relative increase of 20%, the sum must also demonstrate an absolute increase of at least 5 mm. (Note: the appearance of one or more new lesions is also considered progression).

Stable Disease (SD):
Neither sufficient shrinkage to qualify for PR nor sufficient increase to qualify for PD, taking as reference the smallest sum diameters while on study.

As used herein, the term best ORR shall refer to the best overall response rate as per RECIST guideline version 1.1 for the treatment period, where applicable.

As used herein, the term BSA refers to Body Surface Area

As used herein, the term CRC refers to colorectal cancer. The term mCRC shall refer to metastatic colorectal cancer.

As used herein, the terms BRAF mutation-positive patients and KRAS mutation-positive patients shall refer to patients who by genotype testing have been found to harbor either BRAF- or KRAS mutated tumors and/or metastases.

As used herein, the term ctDNA genotype testing shall refer to genotype testing conducted by analyzing a blood or serum sample for cell-free tumor DNA.

### SUMMARY OF INVENTION

Arfolitixorin (Modufolin®) is a new drug developed to increase the efficacy of the cytotoxic agent 5-fluorouracil (5-FU) and as a rescue drug after high-dose methotrexate treatment. Arfolitixorin (Modufolin®), [6R]-5,10-methylenetetrahydrofolate, abbreviated herein as [6R]-5,10-MTHF, needs to be metabolically formed when using the widely used folate-based drugs leucovorin and levoleucovorin. Arfolitixorin (Modufolin®), however, does not require metabolic activation to exert its effect and may therefore be suitable for all patients.

According to the present invention, it has surprisingly been found that patients diagnosed with colorectal cancer and further determined by genotype testing to be either BRAF mutation-positive or KRAS mutation-positive or both BRAF mutation-positive and KRAS mutation-positive, i.e. harboring BRAF mutation- and/or KRAS mutation-positive colorectal cancer tumors, may be treated according to a chemotherapeutic protocol over at least 16 weeks involving i.a. administration of multiple, rapid boluses of [6R]-5,10-MTHF, by which treatment best ORRs (objective response rates) of >50% can be achieved.

Accordingly, in a first aspect of the invention, [6R]-5,10-methylenetetrahydrofolate is provided for use in a human patient in the treatment of solid colorectal cancer tumors, which treatment comprises the following steps:
a) administering a continuous IV infusion containing 85 mg/m² (of BSA) oxaliplatin, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein said human patient has been found by genotype testing to be either BRAF mutation-positive or KRAS mutation-positive or both BRAF mutation-positive and KRAS mutation-positive, and wherein all steps a)-e) are repeated every 2 weeks until termination of the treatment.

In a second aspect of the invention, [6R]-5,10-methylenetetrahydrofolate is provided for use in a human patient in the treatment of solid colorectal cancer tumors, which treatment comprises the following steps:
a) administering a continuous IV infusion containing 180 mg/m² (of BSA) irinotecan, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein said human patient has been found by genotype testing to be either BRAF mutation-positive or KRAS mutation-positive or both BRAF mutation-positive and KRAS mutation-positive, and wherein all steps a)-e) are repeated every 2 weeks until termination of the treatment.

The treatment based on the ARFOX or ARFIRI protocol may in principle be terminated “for any reason”, such as e.g. by a patient decision or a decision taken by the responsible medical person, i.a. due to disease progression or adverse events. Furthermore, the ARFOX or ARFIRI protocol may be interrupted by treatment holidays and the like. Finally the responsible medical person may decide on a fixed number of treatment cycles.

It has also surprisingly been discovered that administration of [6R]-MTHF and 5-FU according to the first or second aspect of the present invention over a treatment period of at least 16 weeks lead to a retardation or prevention of the progression of solid colorectal cancer tumors in a human patient determined to be either KRAS or BRAF mutation-positive.

In a third aspect of the invention [6R]-5,10-methylene-tetrahydrofolate is therefore provided for use in the retardation or prevention of the progression in a human patient of solid colorectal cancer tumors, wherein said human patient has been found by genotype testing to be either BRAF mutation-positive or KRAS mutation-positive or both BRAF mutation-positive and KRAS mutation-positive, which comprises performing and repeating steps a) to e) according to the first or second aspect of the present invention, over a total treatment period of at least 16 weeks.

In a fourth aspect of the invention, there is provided a method for retardation or prevention of the progression in a human of solid colorectal cancer tumors, wherein said human patient has been found by genotype testing to be either BRAF mutation-positive or KRAS mutation-positive or both BRAF mutation-positive and KRAS mutation-positive, which method comprises performing and repeating steps a) to e) according to the first or second aspect of the present invention, over a total treatment period of at least 16 weeks.

### BRIEF DESCRIPTION OF FIGURES

FIG. 1 (adapted from Huang 2018): Table showing a subgroup analysis for the association between KRAS, BRAF and p53 mutations and CRC distant metastasis

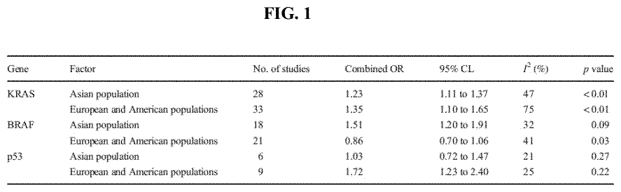

FIG. 2 is a table of all the 31 participating patients in the follow-up study including a status for each patient as regards KRAS and BRAF genotype results.

### DETAILED DESCRIPTION OF THE INVENTION

Arfolitixorin has been in development for a number of years and has been studied in several clinical studies. During one of these studies (the Phase Ulla study ISO-CC-005) it was surprisingly discovered in December 2017 that administration of [6R]-MTHF and 5-FU according to a particular treatment regimen over a treatment period of at least 8 weeks lead to a prevention or retarding of the progression in a human of solid tumors. No statistically significant progression of said solid tumors was observed between 8 and 16 weeks after initiating treatment. These results are discussed i.a. in applicant's international patent application WO 2019/037899 published 28 Feb. 2019. The completion of the study was announced in January 2020. In total, 105 patients were included in the study.

Applicant completed the dose definition part of ISO-CC-005 in March 2018, which evaluated the safety and efficacy of arfolitixorin in patients with mCRC. Shortly after, applicant started two additional treatment groups in 2018 to generate more safety and efficacy data, i.e. the safety extension Cohort #18 (Treatment Arm #4) and Cohort #19 (Treatment Arm #6). The aim was to evaluate as many patients as possible from the additional treatment groups after a treatment period of 16 weeks+.

On 30 Sep. 2020, applicant announced response assessment data from the two safety extension cohorts (31 patients) treated for 16 weeks or longer (press release: 55% Overall Response Rate on the safety extension cohorts of the ISO-CC-005 Phase I/IIa study|BioSpace). The data showed a best overall response rate (ORR) of 55%. These patients had been treated with the selected dose regimen of 120 mg/m2 arfolitixorin and 5-fluorouracil (5-FU) with either irinotecan or oxaliplatin (ARFIRI/ARFOX). Out of the 31 patients, 17 were treated with an ARFOX regimen.

A best ORR of 59% was observed in the ARFOX regimen group versus 50% in the ARFIRI regimen group, despite that 53% of the ARFOX treated patients had a right-sided tumor location and 24% were carrying a BRAF mutation. As mentioned above, in the general CRC population and in historical control first line mCRC Phase III trials, a percentage of approximately 30-40% right-sided tumors are seen and around 10% of the patients carry a BRAF mutation. Both right-sided tumor location and BRAF mutations are historically known as poor prognostic factors and the best ORR in these patient populations in the first line mCRC setting treated with either FOLFOX or FOLFIRI historically generates best ORRs in the range of ˜40% and 15-20% respectively (see eg Loupakis 2018, Van Cutsem 2015 and Tveit 2012).

The average ORR based on pivotal Phase III trials considered in a recent meta-analysis/review indicates that FOLFOX regimens generates 45% best ORR and FOLFIRI regimens generates 40% best ORR in historical non-selected patient population (all-comer), first line mCRC populations (Giuliani 2018).

During a subsequent analysis of the follow-up study results, applicant has now discovered that some of the tested combinations have proven surprisingly effective against KRAS- and BRAF-mutated mCRC tumors. As mentioned hereinabove, these tumor types are particularly aggressive and difficult to treat with cytostatic drugs, and both tumor types are deemed “poor prognostic factors”.

The baseline CRC genotype and sidedness status for the patients enrolled in the two additional treatment groups was collected and summarized as follows:

| Primary tumor location | KRAS status | BRAF status |
|---|---|---|
| Left sided 16 (37%) | Mutant: 17 (40%) | Mutant 9 (21%) |
| Right sided 19 (44%) | Wild type 22 (51%) | Wild type 25 (58%) |
| Rectum 8 (19%) | Unknown 4 (9%) | Unknown 9 (21%) |

Of the 43 patients enrolled in the two safety extension cohorts, 12 patients were either not evaluated with a CT-scan at 8 weeks or beyond 8 weeks of treatment. Of these 12 patients, 4 patients had Stable Disease (SD) and 3 patients had Partial Response (PR) after 8 weeks.

Of the 31 patients actually evaluated at 16 weeks (or more), 13 (42%) had right-sided, 13 (42%) had left-sided, 5 (16%) had rectal CRC, 8 (26%) BRAF-mutated CRC and 11 (35%) KRAS-mutated CRC, which corresponds roughly to the composition of the initial group of 43 patients enrolled in the two safety extension cohorts. In total, 19 patients (61%) had either BRAF-mutated CRC or KRAS-mutated CRC.

The historical objective response rates (ORRs) have been found to be very different for patients with BRAF wild-type (wt) vs. mutant tumors. ORRs were thus found to be app. 20% in patients with BRAF-mutant tumors vs. 50% in those with BRAF (wt) tumors (Li 2020). A similar difference was found for patients with KRAS wild-type (wt) vs. mutant tumors (60% vs. 41%). The latter findings are similar to those of other trials comparing clinical outcomes between patients with KRAS exon 2 (wt) and (mt) tumors (Nakayama 2017). See also Huang 2018, Cefali 2021 and Grassadonia 2019. Based on ORRs alone, the treatment of BRAF- and/or KRAS mutation-positive colorectal cancer patients must therefore be seen as a different, more challenging task than the treatment of colorectal cancer patients in general. As mentioned in the background section of the present application, there are moreover many other reasons for considering BRAF- and/or KRAS mutated CRC tumors for special cases.

As demonstrated above, several clinical studies have found the following approximate ORRs for CRC patients harboring mutations (mt) or wild-type (wt) tumors:

ORR=˜50% for CRC of BRAF (wt) type

ORR=˜20% for CRC of BRAF (mt) type

ORR=˜60%, for CRC of KRAS (wt) type

ORR=˜41%, for CRC of KRAS (mt) type

Returning now to the 31 patients from the follow-up study evaluated at 16 weeks (or more), the “mutation group” of 19 patients upon further analysis contained:

8 cases of CRC with BRAF (mt) type (42%)

11 cases of CRC with KRAS (mt) type (58%)

2 cases of CRC with both KRAS and BRAF mutations

This “mutation group” would be expected to have an overall best ORR reflecting the proportion of BRAF mutation-positive CRC patients+the proportion of KRAS mutation-positive CRC patients, which can be calculated as follows:

Expected ORR_{Mutation group}=Prop_{BRAF(mt)}*ORR_{BRAF(mt)}+Prop_{KRAS(mt)}*ORR_{KRAS(mt)}

This gives, using the historical ORR's discussed above:

Expected ORR_{Mutation group}=42%*20%+58%*41%=32%

However, when assessing the results for the 31-patient group after 16 weeks and beyond of treatment with 120 mg/m2 Arfolitixorin+5-FU+irinotecan or oxaliplatin (the ARFIRI/ARFOX protocols), it was surprisingly found that the 19 patients harbouring either mutated KRAS or BRAF had a best ORR of 53% vs. an expected ORR of ˜32%.

Thus, the ARFIRI/ARFOX treatment protocols have proven surprisingly effective in the treatment of CRC patients having tested BRAF- or KRAS mutation-positive, and much more effective than the FOLFIRI/FOLFOX treatment protocols which employ leucovorin.

Accordingly, in a first aspect of the invention, [6R]-5,10-methylenetetrahydrofolate is provided for use in a human patient in the treatment of solid colorectal cancer tumors, which treatment comprises the following steps:
a) administering a continuous IV infusion containing 85 mg/m² (of BSA) oxaliplatin, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour by
e) administering an IV bolus containing 60 mg/m² (of BSA) [6R]-5,10-methylene-tetrahydrofolate,
   wherein said human patient has been found by genotype testing to be either BRAF mutation-positive or KRAS mutation-positive or both BRAF mutation-positive and KRAS mutation-positive, and wherein all steps a)-e) are repeated every 2 weeks until termination of the treatment.

In a second aspect of the invention, [6R]-5,10-methylenetetrahydrofolate is provided for use in a human patient in the treatment of solid colorectal cancer tumors, which treatment comprises the following steps:
a) administering a continuous IV infusion containing 180 mg/m² (of BSA) irinotecan, followed by
b) administering an IV bolus containing 400 mg/m² (of BSA) 5-fluorouracil, followed by
c) administering an IV bolus containing 60 mg/m² [6R]-5,10-methylenetetrahydrofolate, followed by
d) administering a continuous IV infusion containing 2400 mg/m² 5-fluorouracil over 46 hours±1 hour followed by
e) administering an IV bolus containing 60 mg/m² (of BSA) [6R]-5,10-methylenetetrahydrofolate,
   wherein said human patient has been found by genotype testing to be either BRAF mutation-positive or KRAS mutation-positive or both BRAF mutation-positive and KRAS mutation-positive, and wherein all steps a)-e) are repeated every 2 weeks until termination of the treatment.

Throughout the present application the treatment regimen according to the first aspect is referred to as the “ARFOX” protocol, and the treatment regimen according to the second aspect is referred to as the “ARFIRI” protocol.

The treatment based on the ARFOX or ARFIRI protocol may in principle be terminated “for any reason”, such as e.g. by a patient decision or a decision taken by the responsible medical person, i.a. due to disease progression or adverse events. Furthermore, the ARFOX or ARFIRI protocol may be interrupted by treatment holidays and the like. Finally, the responsible medical person may decide on a fixed number of treatment cycles.

In CRC patients the KRAS and BRAF mutation status is traditionally determined by tumor sample analysis. This requires surgery, and the subsequent analysis (extraction of genomic DNA from the tumor biopsy and analysis for mutations using dPCR) often takes weeks to complete. This creates problems in clinical situations which require urgent treatment based on the mutation status of the patient.

However, several studies the past 5-10 years have demonstrated that genotype testing by analysis of circulating, cell-free tumor DNA (ctDNA) in plasma or serum samples is becoming increasingly accurate and thus important as a non-invasive and fast alternative or supplement to tumor sample analysis. The method is also referred to as “Liquid Biopsy” analysis. Cell-free DNA (cfDNA) is fragmented DNA that is found in the non-cellular blood components.

Among tumor patients, ctDNA is 150-200 base pair fragments that are released by tumor cells into the bloodstream and represents a small fraction of the total cfDNA. Importantly, ctDNA retains epigenetic characteristics and carries tumor-specific mutations that can be detected in peripheral blood (Bi 2020). Analysis of ctDNA in plasma is based on sequencing assays, see eg Finkle 2021.

It was thus reported (Mas 2019) that in a study involving four hundred and twenty-five enrolled mCRC patients, the paired tumor tissue and plasma samples of the patients showed an accuracy of 97.3% (95% CI: 95.2-98.6%) between the BRAF status in plasma and tissue for patients with available paired samples (n=405), and 98.5% (95% CI: 96.4-99.5%) for those with conclusive ctDNA (n=323). The absence of liver metastasis was the main factor associated with inconclusive ctDNA results. In patients with liver metastases, the accuracy was 98.6% (95% CI, 96.5-99.6%).

Similarly, another study (Bachet 2018) involving 329 patients with detectable ctDNA (at least one mutation or one methylated biomarker) showed an accuracy of 94.8% (95% CI, 91.9% to 97.0%) between the RAS mutation status in plasma and tissue. The absence of liver metastases also here was the main clinical factor associated with inconclusive ctDNA results.

Analysis of ctDNA (“liquid biopsy” analysis) is thus deemed an important tool for determining the relevant patient group according to the first or second aspect of the present invention.

Accordingly, in embodiments of the invention, [6R]-5,10-methylene-tetrahydrofolate is provided for use in a human patient in the treatment of solid colorectal cancer tumors, which treatment comprises steps a)-e) according to the first or second aspect of the invention, wherein the human patient has been found either by traditional tumor tissue analysis or in preferred embodiments by ctDNA analysis to be either BRAF mutation-positive or KRAS mutation-positive or both BRAF mutation-positive and KRAS mutation-positive.

During the performance of the follow-up study, 25% of the patients who had been determined to be either BRAF mutation-positive or KRAS mutation-positive or both BRAF mutation-positive and KRAS mutation-positive and treated according to either the ARFOX protocol or ARFIRI protocol were additionally treated with bevacizumab during some time point in study.

In specific embodiments bevacizumab is administered to a human patient during the treatment period according to the first or second aspect. In further embodiments, bevacizumab is administered to a human patient at a dose of 5 mg/kg as an IV infusion every two weeks. In still further embodiments, bevacizumab administration is initiated 8 weeks after initiating treatment.

It has also surprisingly been discovered that administration of [6R]-MTHF and 5-FU according to the first or second aspect of the present invention over a treatment period of at least 16 weeks lead to a retardation or prevention of the progression of solid colorectal cancer tumors in a human patient determined to be either KRAS or BRAF mutation-positive.

In a third aspect of the invention [6R]-5,10-methylene-tetrahydrofolate is therefore provided for use in the retardation or prevention of the progression in a human patient of solid colorectal cancer tumors, wherein said human patient has been found by genotype testing to be either BRAF mutation-positive or KRAS mutation-positive or both BRAF mutation-positive and KRAS mutation-positive, which comprises performing and repeating steps a) to e) according to the first or second aspect of the present invention, over a total treatment period of at least 16 weeks.

In a preferred embodiment of the third aspect, there is provided [6R]-5,10-methylene-tetrahydrofolate for use in the retardation or prevention of the progression of the progression in a human of solid colorectal cancer tumors, wherein said human patient has been found by genotype testing to be either BRAF mutation-positive or KRAS mutation-positive or both BRAF mutation-positive and KRAS mutation-positive, whereby steps a) to e) according to the first or second aspect of the present invention are performed and repeated over a total treatment period of at least 16 weeks, and whereby no statistically significant progression of said solid tumors is observed between 8 and 16 weeks after initiating treatment.

In a fourth aspect of the invention, there is provided a method for retardation or prevention of the progression in a human of solid colorectal cancer tumors, wherein said human patient has been found by genotype testing to be either BRAF mutation-positive or KRAS mutation-positive or both BRAF mutation-positive and KRAS mutation-positive, which method comprises performing and repeating steps a) to e) according to the first or second aspect of the present invention, over a total treatment period of at least 16 weeks.

In a preferred embodiment of the fourth aspect, there is provided a method for retardation or prevention of the progression in a human of solid colorectal cancer tumors, wherein said human patient has been found by genotype testing to be either BRAF mutation-positive or KRAS mutation-positive or both BRAF mutation-positive and KRAS mutation-positive, which method comprises performing and repeating steps a) to e) according to the first or second aspect of the present invention, over a total treatment period of at least 16 weeks, whereby no statistically significant progression of said solid tumors is observed between 8 and 16 weeks after initiating treatment.

In some embodiments of any of the aspects of the invention, 5-fluorouracil (5-FU) is replaced by a fluorinated pyrimidine base such as capecitabine (Xeloda), ie. N4-pentyloxycarbonyl-5′-deoxy-5-fluorocytidine, tegafur, 5-fluoro-pyrimidinone, UFT, doxifluridine, 2′-deoxy-5 fluorouridine, 5′-deoxy-5-fluorouridine, 1-(2′-oxopropyl)-5-FU, and alkyl-carbonyl-5-FU, BOF-A2, ftorafur(TS-1), and S-1.

In preferred embodiments of any of the aspects of the invention, [6R]-5,10-methylenetetrahydrofolate ([6R]-MTHF) is employed as a solid form which is soluble in water, such as a lyophilizate or a salt, optionally stabilized by one or more suitable excipients and/or antioxidants such as citric acid or ascorbic acid or salt forms thereof.

In other preferred embodiments of any of the aspects of the invention the lyophilisate of 6R-MTHF is reconstituted in aqueous media.

In other preferred embodiments of any of the aspects of the invention the lyophilisate of 6R-MTHF is prepared from 6R-MTHF hemisulfate salt.

In other preferred embodiments of any of the aspects of the invention the lyophilisate is prepared from 6R-MTHF hemisulfate salt and trisodium citrate dihydrate.

In preferred embodiments of any of the aspects of the invention, the intravenous bolus administration of steps (b), (c) and (e) occur over of a period of 10 minutes or less.

In preferred embodiments of any of the aspects of the invention, the intravenous bolus administration of steps (b), (c) and (e) occur over of a period of 5 minutes or less.

In preferred embodiments of any of the aspects of the invention, the intravenous bolus administration of steps (b), (c) and (e) occur over of a period of 3 minutes or less.

In preferred embodiments of any of the aspects of the invention, step (c) follows step (b) after a period of 30 minutes±5 minutes.

In preferred embodiments of any of the aspects of the invention, step (d) follows step (c) after a period of less than 60 minutes.

In preferred embodiments of any of the aspects of the invention, step (d) follows step (c) after a period of between 30 and 60 minutes.

### EXAMPLES

Arfolitixorin (former Modufolin®) is a folate-based biomodulator developed by applicant to improve the outcome of a range of antimetabolite treatments used within oncology. One of the therapeutic areas of specific interest included in the development program of Arfolitixorin is as biomodulator of 5-fluorouracil (5-FU) activity in standard treatment regime for advanced, metastatic CRC, such as Stage IV.

The drug substance in Arfolitixorin is [6R]-5,10-MTHF described hereinabove, which is a stable formulation of the naturally occurring diastereoisomer of MTHF. As mentioned in the background section of the present application, Arfolitixorin (Modufolin®), [6R]-5,10-methylenetetrahydrofolate, abbreviated herein as [6R]-5,10-MTHF, needs to be metabolically formed when using the widely used folate-based drugs leucovorin and levoleucovorin. Arfolitixorin (Modufolin®), however, does not require metabolic activation to exert its effect and may be directly involved in the formation of the FdUMP TS ternary complex discussed hereinabove.

Clinical Study ISO-CC-005 was an exploratory, Phase I/II multiple-centre study to be carried out in Stadium IV CRC patients. The study was designed to show clinical relevance for patients by characterizing the tolerability of four Arfolitixorin dose levels (30, 60, 120, and 240 mg/m2) in six different standard clinical settings in the presence of fixed doses of 5-FU alone or in combination with either Oxaliplatin, Irinotecan, or Oxaliplatin and Bevacizumab.

Clinical Study ISO-CC-005 was an exploratory, Phase I/II multiple-centre study to be carried out in Stadium IV CRC patients. The study was designed to show clinical relevance for patients by characterizing the tolerability of four arfolitixorin dose levels (30, 60, 120, and 240 mg/m2) in six different standard clinical settings in the presence of fixed doses of 5-FU alone or in combination with either oxaliplatin, irinotecan, or oxaliplatin and bevacizumab. The below Table shows the initial treatment protocol for the Chemotherapy Agents (Bevacizumab, Oxaliplatin, Irinotecan, and/or 5-FU) and of the Study Drug arfolitixorin (Modufolin®):

TABLE 1

| | | | | | | | 5-FU |
|---|---|---|---|---|---|---|---|
| | | Bevacizumab | | Irinotecan^{#} | | | At approx. |
| | | At approx.\| | Oxaliplatin^{¶} | At approx | | Modufolin [symbol] | 35 minutes |
| | | −180 minutes | At approx. | −60 minutes | 5-FU^{§} [symbol] | At approx. | (46-hour |
| Treatment | | (infusion 30 | −60 minutes | (infusion 30 | At 0 minute | 30 minutes | continuous |
| Arm | Cohort [symbol] | to 90 min) | to 120 min) | to 90 min) | (bolus) | (bolus) [symbol] | infusion) [symbol] |
| Arm 1 | Cohort 1 | N/A | N/A | N/A | 500 mg/m² | 30 mg/m² | N/A |
| | Cohort 2 | N/A | N/A | N/A | 500 mg/m² | 60 mg/m² | N/A |
| | Cohort 8 | N/A | N/A | N/A | 500 mg/m² | 120 mg/m² | N/A |
| | Cohort 9 | N/A | N/A | N/A | 500 mg/m² | 240 mg/m² | N/A |
| Arm 2 | Cohort 4 | N/A | 85 mg/m² | N/A | 500 mg/m² | 30 mg/m² | N/A |
| | Cohort 5 | N/A | 85 mg/m² | N/A | 500 mg/m² | 60 mg/m² | N/A |
| Arm 3 | Cohort 6 | N/A | N/A | 180 mg/m² | 500 mg/m² | 30 mg/m² | N/A |
| | Cohort 7 | N/A | N/A | 180 mg/m² | 500 mg/m² | 60 mg/m² | N/A |
| Arm 4 | Cohort 12 | N/A | 85 mg/m² | N/A | 400 mg/m² | 60 mg/m² [symbol] | 2 400 mg/m² |
| | Cohort 13 | N/A | 85 mg/m² | N/A | 400 mg/m² | 120 mg/m² [symbol] | 2 400 mg/m² |
| | Cohort 14 | N/A | 85 mg/m² | N/A | 400 mg/m² | 240 mg/m² [symbol] | 2 400 mg/m² |
| | Cohort 18a | N/A | 85 mg/m² | N/A | 400 mg/m² | SP2D [symbol] | 2 400 mg/m² |
| | and 18b | | | | | | |
| Arm 5 | Cohort 15 | 5 mg/kg | 85 mg/m² | N/A | 400 mg/m² | 60 mg/m² | 2 400 mg/m² |
| | | | | | | or SP2D ^{a,b} | |
| | Cohort 16 | 5 mg/kg | 85 mg/m² | N/A | 400 mg/m² | 120 mg/m² | 2 400 mg/m² |
| | | | | | | or SP2D ^{a,b} | |
| | Cohort 17 | 5 mg/kg | 85 mg/m² | N/A | 400 mg/m² | 240 mg/m² | 2 400 mg/m² |
| | | | | | | or SP2D ^{a,b} | |
| Arm 6 | Cohort 19a | N/A | N/A | 180 mg/m² | 400 mg/m² | SP2D ^{a, c} | 2 400 mg/m² |
| | and 19b | | | | | | |

Abbreviation:
N/A: not applicable,
SP2D: selected phase 2 dose.
^{¶}The time-point window for Oxaliplatin administration will be expanded to allow infusion times of up to 120 minutes, if necessary
^{#} The time-point window for Irinotecan administration will be expanded to allow infusion times of up to 90 minutes, if necessary.
^{§} The administered bolus 5-FU dose should not surpass the maximum recommended daily dose of 1000 mg, regardless of the body surface area.
* Cohort #3, Cohort #10 and Cohort #11, originally included in earlier versions of this clinical study protocol, have been erased.
^{a} In Treatment Arm #4 (Cohorts #12, #13, #14, #18a, and #18b), Arm #5 (Cohort #15, #16, and #17, if applicable), and Arm #6 (Cohort #19a and #19b) the total Modufolin® dose will be divided into two (2) i.v. bolus injections dispensed approximately 30 and 60 minutes after administration of 5-FU bolus injection (at 0 minute), respectively. The continuous 5-FU infusion will be paused for administration of the second Modufolin® injection.
^{b} The SP2D is the Modufolin® dose level in Treatment Arm #4 (MOFOX) assessed as the dose level with the most favourable profile for following investigation. The SP2D will be the highest Modufolin® dose administered in Treatment Arm #5. [symbol]
indicates data missing or illegible when filed

The tolerability of Arfolitixorin was to be determined by the presence of Dose Limiting Toxicity (DLT) in each of the treatment arms and for each investigated Arfolitixorin dose. For this, the safety of enrolled patients was closely monitored during the study with detailed rules for advancing to next dose cohort(s) or stopping the study.

The study was divided in the Main Study and the Follow-up study. In the Main Study, patients received study treatment with Arfolitixorin during eight (8) weeks. Patients eligible for the Follow-up study could participate until reaching progress, but no longer than 18 months. The Main Study was divided into a dose-finding and a proof-of-concept part. The goal with the Dose-finding Part of the Main Study was to establish the Arfolitixorin dose level assessed as having the most favourable profile, i.e. the selected phase 2 dose (SP2D). The goal with the Proof-of-concept Part of the Main Study was to acquire data on the safety and tolerability of Arfolitixorin at the SP2D dose level in settings equivalent to the two well-established combination therapies FOLFOX (i.e. Oxaliplatin/5-FU/LV) and FOLFIRI (i. e. Irinotecan/5-FU/LV).

Enrolled patients, stadium IV CRC patients, were aware of the relatively poor prognosis of their disease. Those patients who could continue benefitting from treatment with a seemly effective therapy, were offered the possibility to continue study treatment by participating in the Follow-up Study. In the Follow-up Study patients continued to receive the same treatment as assigned during the Main Study period. However, the Investigator could complete the allocated treatment with other therapeutic agents of choice in alignment with standard of care in order to adapt treatment to the patient's specific needs and, in this way, provide optimal care.

Response was measured in short- and long-term assessments. During the Main Study phase, only short-term assessments of tumour response were explored by means of objective response rate (ORR) and early tumour shrinkage (ETS). These assessments were to be used as indicators of prognostic factor in ascertaining earlier non responders and to explore the correlation to other factors such as folate levels, tumour biomarkers, or expression levels of certain key genes.

As mentioned hereinabove, during a subsequent analysis of the follow-up study results applicant has now discovered that some of the tested combinations have proven surprisingly effective in patients determined by genotype testing to be KRAS- and/or BRAF-mutation positive.

The objectives in the follow-up study were to:
To characterise all adverse events (AEs) and clinically significant abnormal laboratory test result changes regardless of attribution during the entire Follow-up Study period.
To evaluate tumour response and disease progression by means of ORR after every response evaluation since treatment allocation in the Main Study and as long patients continue in the Follow-up Study.
To evaluate tumour response and disease progression by means of PFS and TTP since treatment allocation in the Main Study and until the end of patient participation in the Follow-up Study.
To evaluate time-to-death since treatment allocation in the Main Study.
To evaluate the change in tumour biomarker (TK1) levels in blood after every four (4) consecutive cycles of treatment with combination therapy in the subset of patients with available blood samples.

Correlation between tumour biomarkers in blood and treatment response by means of PFS and ORR as per RECIST 1.1 since baseline baseline in the Main Study was determined in the subset of patients with available blood samples. Similarly, the correlation between tumour biomarkers in tumour tissue and treatment response by means of PFS and ORR as per RECIST 1.1 since baseline in the Main Study, was determined in the subset of patients with available tissue biopsy samples.

In the following, the main results of the follow-up study will be discussed.

### Results

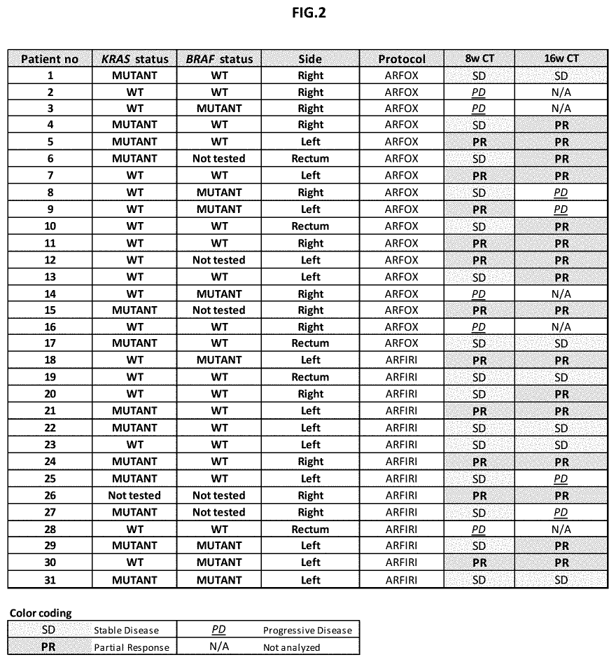

See FIG. 2 for a summarized overview of the 31 participating patients in the Follow-up study. In the following a more detailed, yet anonymized case narrative is given for each patient up to either 8 or 16 weeks treatment, depending on termination.

Patients had been randomized to either the ARFOX or ARFIRI treatment protocol before the follow-up study started, and remained on this protocol for the duration of the study unless otherwise indicated in the below narratives. Patients showing progressive disease (PD) by CT scanning at 8 weeks were not continued in the extension cohorts for another 8 weeks. Patients showing either partial response (PR) or stable disease (SD) at 8 weeks were as a rule continued for 8 more weeks, and assessed again by CT scanning at approximately 16 weeks (or later), i.e. at the 1st follow-up visit. Some patients were also continued for more than 16 weeks, but are not reported here.

The dates for individual events like e.g. genotype testing are shown in square brackets [nn].

### Cases

### Case #1

The patient is a 32-year-old white female randomized for treatment according to the ARFOX protocol (see above). Medical history findings at enrolment are hysterectomy and partial colectomy but no concomitant medication. The primary right sided tumour has been removed but no adjuvant therapy has been given. Genotype testing [2018-05-10] shows patient is KRAS mutant and BRAF wildtype, NRAS wildtype. Genotype testing [2019-04-24] shows patient is microsatelite instability (MSI) stable. Baseline CT [2018-04-30] showed 1 target lesion in the liver (right lobe). At 8 week CT [2018-07-02] the sum of diameter of the target lesion(s) remained (stable disease) and patient was thereby eligible for participation in the follow-up study. At 1st follow-up visit [2018-08-27] CT the sum of diameter of the target lesion(s) decreased with 16% (stable disease). During study participation the following AEs were reported: dry eyes grade 1, fatigue grade 1 and neutrophil count decreased grade 2. The decreased neutrophil count was treated with filgrastim. During the follow-up study bevacizumab was added to the ARFOX treatment.

### Case #2

The patient is a 64-year-old white female randomized for treatment according to the ARFOX protocol (see above). The primary right sided tumour is still in place and no adjuvant therapy has been given. Genotype testing [2018-07-27] shows patient is KRAS, BRAF and NRAS wildtype. Baseline CT [2018-07-02] showed 2 target lesions in the liver parenchyma. At 8 week CT [2018-09-26] the sum of diameter of the target lesion(s) increased with 24% and additional lesions were discovered (progressive disease).

### Case #3

The patient is a 69-year-old white female randomized for treatment according to the ARFOX protocol (see above). No medical history findings and no concomitant medication at enrolment. The primary right sided tumour is still in place and no adjuvant therapy has been given. Genotype testing [2018-09-13] shows patient is KRAS wildtype, BRAF mutant, NRAS wildtype. Genotype testing [2019-02-14] shows patient is MMR stable. Baseline CT [2018-07-04] showed 1 target lesion in the liver (segment IV). At 8 week CT [2018-09-21] the sum of diameter of the target lesion(s) increased with 22% and additional lesions were discovered (progressive disease). No AEs nor concomitant medication reported during study participation.

### Case #4

A 85-year-old white female randomized for treatment according to the ARFOX protocol (see above). The primary right sided tumour has been removed and adjuvant therapy with CAPECITABINE has been given. Genotype testing [2018-09-13] shows patient is KRAS mutant and BRAF and NRAS wildtype. Baseline CT [2018-08-01] showed 1 target lesion in the left lower lobe of the lung. At 8 week CT [2018-10-08] the sum of diameter of the target lesion(s) decreased with 10% (stable disease) and patient consented to participation in the follow-up study. At 1st follow-up visit [2018-11-26] CT the sum of diameter of the target lesion(s) decreased with additional 30% (partial response). During the follow-up study bevacizumab was added to the ARFOX treatment.

### Case #5

The patient is a 69-year-old white female randomized for treatment according to the ARFOX protocol (see above). The primary left sided tumour has been removed but no adjuvant therapy has been given. Genotype testing [2018-09-13] shows patient is KRAS mutant and BRAF and NRAS wildtype. Baseline MM [2018-08-09] showed 1 target lesion in the liver parenchyma. At 8 week CT [2018-11-13] the sum of diameter of the target lesion(s) decreased with 33% (partial responses) and patient consented to participation in the follow-up study. At 1st follow-up visit [2019-03-18] CT the sum of diameter of the target lesion(s) decreased with additional 25% (partial response).

### Case #6

The patient is a 71-year-old white male randomized for treatment according to the ARFOX protocol (see above). The primary rectal tumour is still in place and no adjuvant therapy has been given. Genotype testing [2018-08-24] shows patient is KRAS mutant and MLH1, PMS2 and MSH2 stable. Baseline CT [2018-09-14] showed 1 target lesion in the segment 6/7 of the liver. At 8 week CT [2018-11-05] the sum of diameter of the target lesion(s) decreased with 17% (stable disease) and patient consented to participation in the follow-up study. At 1st follow-up visit [2019-02-15] CT the sum of diameter of the target lesion(s) decrease with additional 25% (partial response).

### Case #7

The patient is a 75-year-old white male randomized for treatment according to the ARFOX protocol (see above). Medical history findings at enrolment are asthma that is treated with Symbicort, and sigmoidectomy. No other concomitant medication. The primary left sided tumour has been removed but no adjuvant therapy has been given. Genotype testing [2018-11-06] shows patient is KRAS, BRAF and NRAS wildtype. Baseline CT [2018-10-12] showed 3 target lesions; 2 in the lung (left and right lower lobe) and 1 in the liver (left lobe). At 8 week CT [2018-12-15] the sum of diameter of the target lesion(s) decreased with 64% (partial responses) and patient consented to participation in the follow-up study. At 1st follow-up visit [2019-02-23] CT the sum of diameter of the target lesion(s) increased with 10% (partial response). During study participation the following AEs were reported: nausea, weight loss and thrombocytopenia, all grade 1. No other concomitant medication during main study in addition to the Symbicort patient had at enrollment in the study. During the follow-up study bevacizumab was added to the ARFOX treatment.

### Case #8

The patient is a 62-year-old white male randomized for treatment according to the ARFOX protocol (see above). The only medical history finding at enrolment is right colectomy and no concomitant medication. The primary right sided tumour has been removed but no adjuvant therapy has been given. Genotype testing [2018-10-16] shows patient is KRAS wildtype, BRAF mutant, NRAS wildtype. Genotype testing [2018-10-24] shows patient is MSI negative. Baseline CT [2018-10-17] showed 5 target lesions in the liver, lung and tumour deposit. At 8 week CT [2018-12-19] the sum of diameter of the target lesion(s) decreased with 6% (stable disease) and patient consented to participation in the follow-up study. At 1st follow-up visit [2019-03-13] CT the sum of diameter of the target lesion(s) increased with 1% but additional lesions were discovered in the lung (progressive disease). During main study SAE pulmonary embolism [2019-12-18] was reported. The following non-serious AEs were reported during main or follow-up: nausea, fatigue, anemia and weight loss. Bevacizumab treatment was initiated during the follow-up study.

### Case #9

The patient is a 61-year-old white female randomized for treatment according to the ARFOX protocol (see above). Medical history findings at enrolment are hyperuricemia and hypertension that are both treated. The primary left sided tumour has been removed but no adjuvant therapy has been given. Genotype testing [2018-10-17] shows patient is KRAS wildtype, BRAF mutant, NRAS wildtype and MSI negative. Baseline CT [2018-10-25] showed 3 target lesions in the liver (segment IV, VI and VII). At 8 week CT [2018-12-27] the sum of diameter of the target lesion(s) decreased with 37% (partial response) and patient consented to participation in the follow-up study. At 1st follow-up visit [2019-03-06] CT the sum of diameter of the target lesion(s) decrease with 15% but additional lesions were discovered (progressive disease). The only AE reported during patient's participation in the study was fatigue grade 1. Bevacizumab treatment was initiated during the follow-up study in addition to the concomitant medication patient had at enrollment in the study.

### Case #10

The patient is a 48-year-old white male randomized for treatment according to the ARFOX protocol (see above). The primary rectal tumour is still in place and no adjuvant therapy has been given. Genotype testing [2018-11-26] shows patient is KRAS, BRAF and NRAS wildtype. Baseline CT+MRI [2018-11-12] showed 3 target lesions; in the liver lobe, in the pericolonic lymph nodes (lymph nodes) and in left pelvis. At 8 week CT [2019-02-09] the sum of diameter of the target lesion(s) decreased with 14% (stable disease) and patient was thereby eligible for participation in the follow-up study. At 1st follow-up visit [2019-05-02] CT the sum of diameter of the target lesion(s) decreases with additional 59% (partial response).

### Case #11

The patient is a 67-year-old white female randomized for treatment according to the ARFOX protocol (see above). Medical history findings at enrolment are an ostomy surgery in the past and an ongoing candida infection, but no concomitant medication. The primary right sided tumour is still in place and no adjuvant therapy has been given. Genotype testing [2018-12-12] shows patient is KRAS and BRAF wildtype, NRAS mutant. Baseline CT [2018-12-16] showed 2 target lesions in the liver (right lobe dorsal lateral). At 8 week CT [2019-02-26] the sum of diameter of the target lesion(s) decreased with 40% (partial response) and patient consented to participation in the follow-up study. At 1st follow-up visit [2019-04-25] CT the sum of diameter of the target lesion(s) decreased with an additional 29% (partial response). Patient terminated study due to Investigators request [2019-05-09] after 1st follow-up visit. During study participation the following AEs were reported: dry skin grade 1, treated with Canoderm, ileostomy infection grade 2, treated with antibiotics, insomnia grade 1 and loss of appetite grade 2. Patient also had a number of AEs related to bone marrow toxicity (neutropenia, leukopenia) with grade ranging from 1-3—treated accordingly with Zarzio, and a couple of occasions of nausea grade 1 despite a number of prophylactic drugs given. During participation patient also developed neuropathy grade 1. During study participation, patient also received thrombosis prophylaxis and constipation prophylaxis. Patient's candida infection [MR] was treated with fluconazole and nystimex.

### Case #12

The patient is a 69-year-old white male randomized for treatment according to the ARFOX protocol (see above). The primary left sided tumour has been removed but no adjuvant therapy has been given. Genotype testing [2018-12-28] shows patient is KRAS and NRAS wildtype. Baseline CT [2018-12-03] showed 3 target lesions; in the part IV of the liver, in left adrenal gland and in lung nodules. At 8 week CT [2019-02-27] the sum of diameter of the target lesion(s) decreased with 36% (partial responses) and patient consented to participation in the follow-up study. At 1st follow-up visit [2019-04-24] CT the sum of diameter of the target lesion(s) decrease with additional 33% (partial response).

### Case #13

Patient 1-41815 (ARFOX) (#13). The patient is a 34-year-old white female randomized for treatment according to the ARFOX protocol (see above). No medical history findings and no concomitant medication at enrolment. The primary left sided tumour has been removed but no adjuvant therapy has been given. Genotype testing [2019-01-31] shows patient is KRAS, BRAF and NRAS wildtype. Baseline Mill [2019-01-02] showed 2 target lesions in the liver (left and right lobe). At 8 week CT [2019-02-28] the sum of diameter of the target lesion(s) decreased with 18% (stable disease) and patient consented to participation in the follow-up study. At 1st follow-up visit [2019-05-13] CT the sum of diameter of the target lesion(s) decreased with an additional 60% (partial response). No AEs nor concomitant medication reported during main study, but panitimumab was added to the ARFOX treatment during the follow-up study.

### Case #14

The patient is a 53-year-old white male randomized for treatment according to the ARFOX protocol (see above). Medical history finding at enrolment is depression, which is treated with Sobril. Other concomitant medication at enrolment is treatment of pain, heartburn, rhinit as well as constipation and thrombosis prophylaxes. The primary right sided tumour is still in place and no adjuvant therapy has been given. Genotype testing [2019-01-09] shows patient is KRAS wildtype, BRAF mutant, NRAS wildtype and MSS negative. Baseline CT [2019-01-02] showed 3 target lesions in the liver (segment IV and VII) and lymph node. At 8 week CT [2019-03-27] the sum of diameter of the target lesion(s) increased with 29% and additional lesions were discovered (progressive disease).

### Case #15

The patient is a 68-year-old white male randomized for treatment according to the ARFOX protocol (see above). The primary right sided tumour has been removed but no adjuvant therapy has been given. Genotype testing [2019-01-31] shows patient is KRAS mutant and MSS negative. Baseline CT [2019-02-21] showed 2 target lesions; in segment 6/7 and segment 8 of the liver. At 8 week CT [2019-04-15] the sum of diameter of the target lesion(s) decreased with 53% (partial responses) and patient was thereby eligible for participation in the follow-up study. At 1st follow-up visit [2019-06-12] CT the sum of diameter of the target lesion(s) decrease with additional 30% (partial response).

### Case #16

The patient is a 70-year-old white male randomized for treatment according to the ARFOX protocol (see above). The primary right sided tumour has been removed but no adjuvant therapy has been given. Genotype testing [2018-09-26] shows patient is MSI stable (microsatellite instability absent). Genotype testing [2020-02-04] shows patient is KRAS, BRAF and NRAS wildtype. Baseline CT [2019-03-06] showed 1 target lesion in the liver. At 8 week CT [2019-06-07] the sum of diameter of the target lesion(s) remained but additional lesions were discovered in the lung (progressive disease).

### Case #17

The patient is a 66-year-old white female randomized for treatment according to the ARFOX protocol (see above). The primary rectal tumour is still in place and no adjuvant therapy has been given. Genotype testing [2019-01-08] shows patient is KRAS mutant and BRAF and NRAS wildtype. Baseline CT [2019-03-07] showed 2 target lesions; in the left lower lung lobe and in the right upper lung lobe. At 8 week CT [2019-05-17] the sum of diameter of the target lesion(s) decreased with 12% (stable disease) and patient consented to participation in the follow-up study. At 1st follow-up visit [2019-07-29] CT the sum of diameter of the target lesion(s) remained (stable disease).

### Case #18

The patient is a 68-year-old white male randomized for treatment according to the ARFIRI protocol (see above). Medical history findings at enrolment are depression, gastroesophageal reflux and insomnia which are treated accordingly. Other medical history findings are untreated atrial fibrillation grade 2 and back pain. Patient also has liver surgery and hemicolectomy reported as medical history. The primary left sided tumour has been removed and adjuvant therapy with FOLFOX [EOT 2017 05] has been given. Genotype testing shows patient is KRAS wildtype, BRAF mutant, NRAS wildtype. Baseline MRI [2018-03-06] showed 1 target lesion in the liver (right dorsal). At 8 week MRI [2018-05-28] the sum of diameter of the target lesion(s) decreased with 30% (partial response) and patient consented to participation in the follow-up study. At 1st follow-up visit [2018-08-15] MM the sum of diameter of the target lesion(s) decreased with an additional 40% (partial response). During the study, the patient had severe problems with GI related toxicity such as nausea and vomiting. Patient was allowed to try the following 5-FU bolus regimen (irinotecan 180 mg/m2 on day 1, bolus 5-FU 500 mg/m2 and arfolitixorin 60 mg/m2 on day 1 and 2) during the follow-up study without any significant change of toxicity. Patient also reported a number of occasions of fatigue grade 1-2 during study participation and initially one episode of paroxysmal atrial fibrillation grade 3 and a month later atrial fibrillation grade 3—both reported to be related to the study drug by the investigator. During follow-up study the patient twice receives radiotherapy due to AE costal pain.

### Case #19

The patient is a 65-year-old white female randomized for treatment according to the ARFIRI protocol (see above). The primary rectal tumour is still in place and adjuvant therapy with CAPECITABINE-OXALIPLATIN [EOT 2016-01-29] has been given. Genotype testing [2018-03-22] shows patient is KRAS, BRAF and NRAS wildtype. Baseline CT [2018-03-27] showed 1 target lesion in the pelvis (cervix, near rectal stump). At 8 week CT [2018-06-22] the sum of diameter of the target lesion(s) remained (stable disease) and patient was thereby eligible for participation in the follow-up study. At 1st follow-up visit [2018-09-03] CT the sum of diameter of the target lesion(s) remained (stable disease). Patient terminated study due to AE ‘Thromboembolic event’ [2018-09-03] after 1st follow-up visit. During the follow-up study bevacizumab was added to the ARFIRI treatment.

### Case #20

The patient is a 74-year-old white male randomized for treatment according to the ARFIRI protocol (see above). Medical history findings at enrolment are hypertension that is treated accordingly, hyperlipidemia and a right hemicolectomy. Patient is treated with Salospir and Placol as cardiovascular prevention. The primary right sided tumour has been removed and adjuvant therapy with CAPECITABINE-OXALIPLATIN [EOT 2017-UNK-UNK] has been given. Genotype testing [2018 04] shows patient is KRAS, BRAF and NRAS wildtype. Baseline CT [2018-05-14] showed 5 target lesions in the lung (right and left lobe), abdomen and abdominal aorta. At 8 week CT [2018-07-10] the sum of diameter of the target lesion(s) decreased with 27% (stable disease) and patient was thereby eligible for participation in the follow-up study. At 1st follow-up visit [2018-08-31] CT the sum of diameter of the target lesion(s) decreased with an additional 10% (partial response). Patient terminated study due to PI decision due to maximum clinical benefit [2018-10-05]. The only AE reported was a diarrhea grade 1 during follow-up study and during the follow-up study bevacizumab was added to the ARFIRI treatment. No other concomitant medication in addition to the concomitant medication patient had at enrollment in the study.

### Case #21

The patient is a 67-year-old white male randomized for treatment according to the ARFIRI protocol (see above). The primary left sided tumour is still in place and no adjuvant therapy has been given. Genotype testing [2018-05-22] shows patient is KRAS mutant, BRAF wildtype and MLH1, PMS2, MSH2 and MSH6 stable. Baseline CT [2018-04-16] showed 3 target lesions in the liver; 1 in left lobe apical and 2 in right lobe. At 8 week CT [2018-07-09] the sum of diameter of the target lesion(s) decreased with 42% (partial responses) and patient consented to participation in the follow-up study. At 1st follow-up visit [2018-10-17] CT the sum of diameter of the target lesion(s) decreased with additional 43% (partial response).

### Case #22

The patient is a 58-year-old white male randomized for treatment according to the ARFIRI protocol (see above). The primary left sided tumour has been removed but no adjuvant therapy has been given. Genotype testing [2017-11-28] shows patient is KRAS mutant and BRAF and NRAS wildtype. Baseline CT [2018-07-23] showed 2 target lesions in the lung; right lower lobe and lymphnode. At 8 week CT [2018 26] the sum of diameter of the target lesion(s) remained (stable disease) and patient consented to participation in the follow-up study. At 1st follow-up visit [2018-12-13] CT the sum of diameter of the target lesion(s) remained (stable disease). During the follow-up study bevacizumab was added to the ARFIRI treatment.

### Case #23

The patient is a 68-year-old white male randomized for treatment according to the ARFIRI protocol (see above). Medical history findings at enrolment are hypertension and restless legs that are treated accordingly. At enrolment, a Peripherally inserted central catheterization is done. No other concomitant medication. The primary left sided tumour has been removed but no adjuvant therapy has been given. Genotype testing [2018-08-10] shows patient is KRAS wildtype, BRAF wildtype and MLH1, PMS2, MSH2 and MSH6 stable. Baseline MRI [2018-07-06] showed 2 target lesions in the liver (segment 1 and 7). At 8 week MM [2018-10-01] the sum of diameter of the target lesion(s) decreased with 13% (stable disease) and patient consented to participation in the follow-up study. At 1st follow-up visit [2019-01-10] MRI the sum of diameter of the target lesion(s) decrease with an additional 20% (stable disease). During the first 8 weeks of study participation the following AEs were reported: hypotension grade 2, insomnia grade 1 and worsening of restless legs (grade 1) that was reported as medical history. Beside treatment of the AEs reported, patient also received constipation prophylaxis, thrombosis prophylaxis and nausea prophylaxis.

### Case #24

The patient is a 58-year-old white female randomized for treatment according to the ARFIRI protocol (see above). Medical history findings at enrolment are depression and pain that are treated accordingly. Other medical history findings are twisted ovarian cyst, struma [goitre], anorexia, rash, fatigue and dry mouth. Medication for rash is prescribed at enrolment. The primary right sided tumour has been removed but no adjuvant therapy has been given. Genotype testing [2018-07-31] shows patient is KRAS mutant and BRAF wildtype, NRAS wildtype. Baseline CT [2018-08-22] showed 2 target lesions in retroperitoneal lymph node. At 8 week CT [2018-10-22] the sum of diameter of the target lesion(s) decreased with 47% (partial response) and patient consented to participation in the follow-up study. At 1st follow-up visit [2018-12-17] CT the sum of diameter of the target lesion(s) decreased with an additional 32% (partial response). Dizziness grade 1 and nausea grade 1 are reported at almost every treatment cycle, and nausea is treated with both oral an i.v. nausea prophylaxis. Patient also reports several episodes of epistaxis grade 1. Dry skin grade 1 is reported a couple of times and so is pain grade 1, lasting for several weeks at a time. During follow-up study oral mucositis lasting for more than a month is reported, treated with chamomile flower tea.

### Case #25

The patient is a 65-year-old white male randomized for treatment according to the ARFIRI protocol (see above). The primary left sided tumour has been removed but no adjuvant therapy has been given. Genotype testing [2017-10-12] shows patient is KRAS mutant and BRAF wildtype. Genotype testing [2017-10-13] shows patient is MSI negative. Baseline CT [2018-08-23] showed 1 target lesion in segment VI of the liver. At 8 week CT [2018-10-31] the sum of diameter of the target lesion(s) remained (stable disease) and patient consented to participation in the follow-up study. At 1st follow-up visit [2019-01-09] CT the sum of diameter of the target lesion(s) remained but additional lesions were discovered (progressive disease).

### Case #26

The patient is a 63-year-old white male randomized for treatment according to the ARFIRI protocol (see above). The primary right sided tumour has been removed but no adjuvant therapy has been given. No genotype testing performed. Baseline CT [2018-10-29] showed 2 target lesions; left ventral (gland) and left aorta (gland). At 8 week CT [2018-12-18] the sum of diameter of the target lesion(s) decreased with 54% (partial response) and patient consented to participation in the follow-up study. At 1st follow-up visit [2019-02-20] CT the sum of diameter of the target lesion(s) decreased with additional 8% (partial response).

### Case #27

The patient is a 45-year-old white male randomized for treatment according to the ARFIRI protocol (see above). The primary right sided tumour has been removed and adjuvant therapy with FLOX [EOT 2017-06-29] has been given. Genotype testing [2018-08-06] shows patient is KRAS mutant and MLH1, PMS2, MSH2 and MSH6 stable. Baseline CT [2018-12-07] showed 1 target lesion the right lower lobe of the lung, ventral. At 8 week CT [2019-02-11] the sum of diameter of the target lesion(s) remained (stable disease) and patient consented to participation in the follow-up study. At 1st follow-up visit [2019-05-20] CT the sum of diameter of the target lesion(s) increased with 50% (progressive disease).

### Case #28

The patient is a 46-year-old Asian female randomized for treatment according to the ARFIRI protocol (see above). The primary rectal tumour is still in place and no adjuvant therapy has been given. Genotype testing [2018-11-16] shows patient is KRAS and BRAF wildtype and NRAS mutant. Baseline CT [2018-12-27] showed 2 target lesions in the liver. At 8 week CT [2019-04-08] the sum of diameter of the target lesion(s) increased with 80% (progressive disease).

### Case #29

Patient 1-61918 (ARFIRI) (#29). The patient is a 72-year-old white female randomized for treatment according to the ARFIRI protocol (see above). Medical history finding at enrolment is (mild) hypertension treated with hydrochlorothiazide. No other medical history finding nor concomitant medication. The primary left sided tumour has been removed but no adjuvant therapy has been given. Genotype testing [2019-03-01] shows patient is KRAS and BRAF mutant, NRAS mutant. Genotype testing [2019-12-12] shows patient is MSI stable (microsatellite instability absent). Baseline CT [2019-03-19] showed 1 target lesion in the lung (left upper lobe). At 8 week CT [2019-05-11] the sum of diameter of the target lesion(s) remained (stable disease) and patient consented to participation in the follow-up study. At 1st follow-up visit [2019-07-19] CT the sum of diameter of the target lesion(s) decreased with 44% (partial response). No AEs and no other concomitant medication in addition to the concomitant medication patient had at enrollment in the study.

### Case #30

The patient is a 68-year-old white female randomized for treatment according to the ARFIRI protocol (see above). Medical history findings at enrolment are hypercholesterolemia, paroxysmal atrial tachycardia and chronic respiratory failure which are treated accordingly. The primary left sided tumour has been removed but no adjuvant therapy has been given. Genotype testing [2019-04-11] shows patient is BRAF mutant and MSI stable (microsatellite instability absent). Genotype testing [2019-04-16] shows patient is KRAS wildtype. Genotype testing [2019-04-19] shows patient is NRAS wildtype. Baseline CT [2019-04-03] showed 3 target lesions in the liver (right lobe) and lung (right lobe). At 8 week CT [2019-06-04] the sum of diameter of the target lesion(s) decreased with 54% (partial response) and patient consented to participation in the follow-up study. At 1st follow-up visit [2019-08-02] CT the sum of diameter of the target lesion(s) decreased with an additional 39% (partial response). Two AEs are reported during follow-up study; diarrhea and fatigue grade 2. No other concomitant medication in addition to the concomitant medication patient had at enrollment in the study.

### Case #31

The patient is a 52-year-old white female randomized for treatment according to the ARFIRI protocol (see above). No medical history findings and no concomitant medication at enrolment. The primary left sided tumour has been removed but no adjuvant therapy has been given. Genotype testing [2019-04-25] shows patient is KRAS and BRAF mutant, NRAS wildtype. Genotype testing [2019-04-19] shows patient is MSI stable (microsatellite instability absent). Baseline CT [2019-03-13] showed 2 target lesions in the lung (left and right lower lobe). At 8 week CT+MRI [2019-06-06] the sum of diameter of the target lesion(s) decreased with 15% (stable disease) and patient consented to participation in the follow-up study. At 1st follow-up visit [2019-07-29] CT the sum of diameter of the target lesion(s) increased with 3% (stable disease). Patient terminated study due to patient's request [2019-08-06]. No AEs nor concomitant medication reported during study participation.

### REFERENCES

1. F Caputo et al. BRAF-Mutated Colorectal Cancer: Clinical and Molecular Insights. Int. J. Mol. Sci. 2019, 20, 5369
2. F Bray, et al. Global cancer statistics 2018: GLOBOCAN estimates of incidence and mortality worldwide for 36 cancers in 185 countries. CA Cancer J. Clin. 2018, 68, 394-424.
3. American Cancer Society. Cancer Facts & Figures; American Cancer Society: Atlanta, Ga., USA, 2016.
4. C S Fuchs et al. Randomized, controlled trial of irinotecan plus infusional, bolus, or oral fluoropyrimidines in first-line treatment of metastatic colorectal cancer: Results from the BICC-C Study. J. Clin. Oncol. 2007, 25, 4779-4786.
5. J Tabernero et al. Unmet Medical Need in Patients with Metastatic Colorectal Cancer with BRAF V600E Mutations: A Review. EMJ Oncol. 2020; 8[Suppl 3]:2-14.
6. B Baran et al. Difference Between Left-Sided and Right-Sided Colorectal Cancer:

A Focused Review of Literature. Gastroenterol Res. 2018; 11(4):264-273
7. CM Ribic et al. Tumor Microsatellite-Instability Status as a Predictor of Benefit from Fluorouracil-Based Adjuvant Chemotherapy for Colon Cancer. N Engl J Med. 2003; 349(3):247-257
8. BM Wolpin et al. Systematic treatment of colorectal cancer. Gastroenterol. 2008; 134(5):1296 1310
9. H Brenner et al. Colorectal cancer. Lancet. 2014; 383(9927):1490-1502.
10. Peng et al. Right- and left-sided stage III colon cancers present different prognostic outcomes of oxaliplatin-based adjuvant chemotherapy after curative resection. Cancer Management and Research 2018:10
11. FA Sinicrope et al. Prognostic Impact of Deficient DNA Mismatch Repair in Patients With Stage III Colon Cancer From a Randomized Trial of FOLFOX-Based Adjuvant Chemotherapy. J Clin Oncol. 2013; 31(29):3664-3672.
12. C Cremolini et al. When to Use Triplet Chemotherapy as First-Line Treatment in Metastatic Colorectal Cancer. Clinical Advances in Hematology & Oncology August 2019—Volume 17, Issue 8
13. C Cremolini et al. Individual Patient Data Meta-Analysis of FOLFOXIRI Plus Bevacizumab Versus Doublets Plus Bevacizumab as Initial Therapy of Unresectable Metastatic Colorectal Cancer. Journal of Clinical Oncology 38(28) August 2020
14. F Loupakis et al. Impact of primary tumour location on efficacy of bevacizumab plus chemotherapy in metastatic colorectal cancer. BJC (2018) 119:1451-1455
15. Van Cutsem et al. Fluorouracil, leucovorin, and irinotecan plus cetuximab treatment and RAS mutations in colorectal cancer. J. Clin Oncol. (2015) 33:692-700
16. KM Tveit et al. Phase III trial of cetuximab with continuous or intermittent fluorouracil, leucovorin, and oxaliplatin (Nordic FLOX) versus FLOX alone in first-line treatment of metastatic colorectal cancer: the NORDIC-VII study J. Clin Oncol. (2012) 30(15):1755-1762)
17. M SIDERIS et al. BRAF V600E Mutation in Colorectal Cancer Is Associated with Right-sided Tumours and Iron Deficiency Anaemia. ANTICANCER RESEARCH 35: 2345-2350 (2015)
18. P Ross Right versus left-sided colon cancer: Is it time to consider these as different diseases? AIMS Medical Science, 5(3): 303-315 (2018)
19. D Hanna, How We Treat Left-Sided vs Right-Sided Colon Cancer. Clinical Advances in Hematology & Oncology Volume 18, Issue 5 May 2020
20. H Taniguchi et al. Tumor Location Is Associated With the Prevalence of Braf And Pik3ca Mutations in Patients with Wild-Type Ras Colorectal Cancer: A Prospective Multi-Center Cohort Study in Japan. Translational Oncology 13 (2020) 100786
21. Li et al. Gastroenterology Report, 8(3), 2020, 192-205
22. Greystoke et al. How Many Diseases Are Colorectal Cancer? Gastroenterology Research and Practice, Vol. 2012, p. 1-12
23. Gomez et al. Anatomical distribution of colorectal cancer over a 10 year period in a district general hospital: is there a true “rightward shift?” Postgrad Med J 2004; 80:667-669
24. Grassadonia et al. Impact of primary tumor location in patients with RAS wild-type metastatic colon cancer treated with first-line chemotherapy plus anti-EGFR or anti-VEGF monoclonal antibodies: a retrospective multicenter study. Journal of Cancer 2019, Vol. 10, 5926
25. Recio-Boiles A, et al. Rectal Cancer. [Updated 2020 Dec. 17]. In: StatPearls [Internet]. Treasure Island (FL): StatPearls Publ. 2021. Available from: https://www.ncbi.nlm.nih.gov/books/NBK493202/
26. Sun, D c., et al. KRAS mutation and primary tumor location do not affect efficacy of bevacizumab-containing chemotherapy in stage IV colorectal cancer patients. Sci Rep 7, 14368 (2017).
27. Koumarianou A et al., Implications of KRAS status in first line chemotherapy with bevacizumab in advanced colorectal cancer: A phase IV study of Hellenic Oncology Research Group (HORG). Journal of Clinical Oncology 2018 36:15 supp
28. Sanford N et al., Early-onset colorectal cancer: more than one side to the story. COLORECTAL CANCER VOL. 9, NO. 3 (2020)
29. Burge M et al., First-line therapy for metastatic colorectal cancer: Current perspectives and future directions. Asia-Pac J Clin Oncol. 2019; 15(Suppl. 1): 3-14.
30. Giuliani et al., Int J Colorectal Dis. 2018 November; 33(11): 1505-1516 (2018)
31. M Cefali et al., Research progress on KRAS mutations in colorectal cancer. J Cancer Metastasis Treat 2021; 7:26
32. D Huang et al., Mutations of key driver genes in colorectal cancer progression and metastasis. Cancer and Metastasis Reviews (March 2018)
33. I Nakayama et al., Retrospective study of RAS/PIK3CA/BRAF tumor mutations as predictors of response to first-line chemotherapy with bevacizumab in metastatic colorectal cancer patients. BMC Cancer (2017) p 1-9
34. L Mas et al., BRAF Mutation Status in Circulating Tumor DNA from Patients with Metastatic Colorectal Cancer: Extended Mutation Analysis from the AGEO RASANC Study. Cancers 2019, 11, 998; doi:10.3390/cancers1107099.
35. J B Bachet et al., RAS mutation analysis in circulating tumor DNA from patients with metastatic colorectal cancer: the AGEO RASANC prospective multicenter study. Ann Oncol. 2018 May 1; 29(5):1211-1219. doi: 10.1093/annonc/mdy061.
36. A Venook et al., Impact of primary (1°) tumor location on overall survival (OS) and progression-free survival (PFS) in patients (pts) with metastatic colorectal cancer (mCRC): Analysis of CALGB/SWOG 80405 (Alliance). Journal of Clinical Oncology 34, no. 15_suppl (May 20, 2016) 3504-3504.
37. Y Liu et al., Analysis of KRAS mutations in circulating tumor DNA and colorectal cancer tissue. Biotech Histochem 2021 July; 96(5):376-383. doi:10.1080/10520295.2020.1810775
38. H Bando et al., A multicentre, prospective study of plasma circulating tumour DNA test for detecting RAS mutation in patients with metastatic colorectal cancer. British Journal of Cancer (2019) 120:982-986; https://doi.org/10.1038/s41416-019-045′7-y.
39. F Bi et al., Circulating tumor DNA in colorectal cancer: opportunities and challenge. Am J Transl Res 2020; 12(3):1044-105.
40. J D Finkle et al., Validation of a liquid biopsy assay with molecular and clinical profiling of circulating tumor DNA. npj Precis. Onc. 5, 63 (2021). https://doi.org/10.1038/s41698-021-00202-2

What is claimed is:

1. A method of treating a human patient diagnosed with a solid tumor colorectal cancer or metastatic colorectal cancer comprising the following steps:
a) selecting a human patient who is either BRAF mutation positive or KRAS mutation-positive or both BRAF mutation positive and KRAS mutation positive,
b) administering to said patient a continuous intravenous (IV) infusion of a pharmaceutical composition comprising either
i. 85 mg/m² (of BSA) oxaliplatin, or
ii. 180 mg/m² (of BSA) irinotecan,
followed by
c) administering to said patient an IV bolus of a pharmaceutical composition comprising 400 mg/m² (of BSA) 5-fluorouracil (5-FU), followed by
d) administering to said patient an IV bolus of a pharmaceutical composition comprising 60 mg/m² [6R]-5,10-methylenetetrahydrofolate (6R-MTHF), followed by
e) administering to said patient a continuous IV infusion of a pharmaceutical composition comprising 2400 mg/m² 5-FU over 46 hours±1 hour followed by
f) administering to said patient an IV bolus of a pharmaceutical composition comprising 60 mg/m² (of BSA) 6R-MTHF.

2. The method of claim 1, wherein steps b)-f) are repeated every 2 weeks for a total treatment period of at least 16 weeks.

3. The method of claim 1, wherein steps b)-f) are repeated every 2 weeks until termination of the treatment.

4. The method of claim 1, wherein genotype testing has shown that the patient is KRAS mutation-positive.

5. The method of claim 1, wherein genotype testing has shown that the patient is BRAF mutation-positive.

6. The method of claim 1, wherein genotype testing has shown that the patient is both KRAS mutation-positive and BRAF mutation-positive.

7. The method of claim 1, further comprising administering to said patient during the treatment period a pharmaceutical composition comprising bevacizumab.

8. The method of claim 7, wherein the pharmaceutical composition comprising 5 mg/kg of the pharmaceutical composition comprising bevacizumab is administered as an IV infusion every two weeks.

9. The method of claim 8, wherein bevacizumab administration begins 8 weeks after initiating step b) of the treatment.

10. The method of claim 8, wherein bevacizumab administration begins prior to initiating step b) of the treatment.

11. The method of claim 1, wherein the 6R-MTHF is reconstituted from a lyophilisate prior to administration.

12. The method of claim 11, wherein the lyophilisate is reconstituted in aqueous media.

13. The method of claim 11, wherein the lyophilisate of 6R-MTHF is prepared from 6R-MTHF hemisulfate salt.

14. The method of claim 13, wherein the lyophilisate is prepared from 6R-MTHF hemisulfate salt and trisodium citrate dihydrate.

15. The method of claim 1, wherein the pharmaceutical composition comprising 6R-MTHF further comprises citric or ascorbic acid or salts thereof.

16. The method of claim 1, wherein the 6R-MTHF has a diastereomeric purity of >98% d.e.

17. The method of claim 2, wherein said method retards or inhibits progression of said solid tumors.

18. The method of claim 17, wherein said method produces no statistically significant progression of said solid tumors up to at least 16 weeks after initiating treatment.

19. The method of claim 1, wherein the intravenous bolus administration of steps (c), (d) and (f) occur over of a period of 10 minutes or less.

20. The method of claim 1, wherein the intravenous bolus administration of step (c), (d) or (f) occur over a period of 5 minutes or less.

21. The method of claim 1, wherein the intravenous bolus administration of step (c), (d) or (f) occur over a period of 3 minutes or less.

22. The method of claim 1, wherein step (d) follows step (c) after a period of 30 minutes±5 minutes.

23. The method of claim 1, wherein step (e) follows step (d) after a period of less than 60 minutes.

24. The method of claim 1, wherein step (e) follows step (d) after a period of between 30 and 60 minutes.
